(19) United States
(12) Patent Application Publication
(10) Pub. No.: US 2010/0113602 A1
(43) Pub. Date: May 6, 2010

(54) USE OF HISTONE DEACETYLASE INHIBITORS FOR THE TREATMENT OF CENTRAL NERVOUS SYSTEM METASTASES

(75) Inventors: Diane Palmieri, Germantown, MD (US); Patricia Steeg, Laurel, MD (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6731, US

(73) Assignee: The United States of America,as represented by the Secretary,Department of Health and Human Services, Bethesda, MD (US)

(21) Appl. No.: 12/528,597

(22) PCT Filed: Feb. 27, 2008
(86) PCT No.: PCT/US08/55149

§ 371 Date: Sep. 28, 2009

Related U.S. Application Data
(60) Provisional application No. 60/891,856, filed on Feb. 27, 2007.

(51) Int. Cl. A61K 31/167; A61P 35/00

(52) U.S. Cl. 514/619

(57) ABSTRACT

Disclosed is a method of treating a localized carcinoma central nervous system (CNS) metastasis of extra-CNS origin, the method comprising systemically administering an effective amount of a histone deacetylase (HDAC) inhibitor (HDI) to a subject in need of treatment for the localized carcinoma CNS metastasis of extra-CNS origin. The HDI can be any HDI capable of crossing the blood-brain barrier (BBB) such as vorinostat. The localized carcinoma CNS metastasis of extra-CNS origin can be a localized carcinoma brain metastasis. The localized carcinoma brain metastasis can originate in the breast. The CNS metastasis treated can be a micrometastasis, a brain tumor, or an intervening stage of brain cancer.

### CROSS-REFERENCE TO RELATED APPLICATIONS

This patent application claims the benefit of U.S. Provisional Patent Application No. 60/891,856, filed Feb. 27, 2007, which is incorporated by reference.

### BACKGROUND OF THE INVENTION

An increased incidence of brain metastases has followed the increased survival of primary and metastatic systemic cancers made possible by improved systemic therapies. Approximately 10-20% of women with metastatic breast cancer will develop clinically apparent brain metastases. The median survival after diagnosis of a central nervous system (CNS) metastasis is approximately one year. Additionally, the incidence of CNS metastases at autopsy range from 18-30%. Relatively few treatment options are available for women with metastatic breast cancer and particularly with a CNS metastasis. Accordingly, there is a desire for a method for treating a CNS metastasis especially carcinoma brain metastases originating outside of the CNS.

### BRIEF SUMMARY OF THE INVENTION

The present invention provides a method of treating a localized carcinoma central nervous system (CNS) metastasis of extra-CNS origin, the method comprising systemically administering an effective amount of a histone deacetylase (HDAC) inhibitor (HDI) to a subject in need of treatment for the localized carcinoma CNS metastasis of extra-CNS origin. The HDI can be any HDI capable of crossing the blood-brain barrier (BBB) such as vorinostat. The localized carcinoma CNS metastasis of extra-CNS origin can be a localized carcinoma brain metastasis. The localized carcinoma CNS metastasis can originate in one or more organs such as the lung, breast, colon, liver, and prostate. The subject can be a former or current cancer patient, and may or may not have been previously treated for cancer. The subject may have had one or more non-CNS localized metastases. The subject treated with the disclosed method can be administered vorinostat alone or in combination with one or more additional drugs. The subject treated with the method of the invention can be administered vorinostat together with a radiation treatment regimen. The CNS metastasis treated can be a micrometastasis, a brain tumor, or an intervening stage of brain cancer.

### BRIEF DESCRIPTION OF THE DRAWING

FIG. 1 is a graph depicting the percentage of cells stained for Ki67 in large metastases (>50 microns²) in vehicle- and vorinostat-(SAHA-) treated mouse brains. The horizontal bars indicate the mean.

### DETAILED DESCRIPTION OF THE INVENTION

A method of treating a localized carcinoma central nervous system (CNS) metastasis of extra-CNS origin is provided, the method comprising systemically administering an effective amount of a histone deacetylase (HDAC) inhibitor (HDI) to a subject in need of treatment for the localized carcinoma CNS metastasis of extra-CNS origin. The metastasis treated is “localized” in that it is located somewhere in the CNS. In some embodiments, it is located in brain. In those embodiments where the CNS metastatis is located in the brain, the metastasis can be located in one or more of the brain parenchyma, the leptomeninges, the cerebrum, the cerebellum, and the brain stem (including the midbrain, medulla oblongata and the pons). When the metastasis is located in the leptomeninges, it can be located in the pia, the arachnoid, the cerebral spinal fluid (CSF)-filled space between the pia and arachnoid membranes, the dura matter, the space between the arachnoid and dura matter, and any combination thereof. In some embodiments, the metatstasis is localized in the spinal cord. A metastasis in the spinal cord can include a bone metastasis. In certain embodiments, the metastatis is located in the cranial nerves. The metastasis treated is larger than a single cell that has localized to the brain and is at least a micrometastasis. The CNS metastasis can comprise a micrometastasis, a brain tumor, or an intervening stage of brain cancer. More than one metastasis can be present in the CNS and the multiple metastases need not be located in the same part of the CNS. The metastasis can be characterized in its microvessel density and aspects of angiogenesis. The subject treated can have one or more primary cancers of the brain or metastases originating in the brain or elsewhere in the CNS in addition to one or more localized carcinoma CNS metastasis of extra-CNS origin. The subject treated can have one or more non-carcinoma CNS metastasis such as a melanoma, lymphoma or sarcoma, e.g., an osteosarcoma. The subject treated in accordance with the invention must have at least one localized carcinoma CNS metastasis. However, a metastasis need not have been detected prior to or concurrent with treatment. A recognized increased susceptibility to a localized carcinoma CNS metastasis can also be relied upon. The carcinoma CNS metastasis can have one or more difference in gene expression compared to the primary systemic carcinoma from which it is derived.

The HDI employed in accordance with the method can be any HDI capable of crossing the blood-brain barrier (BBB) such as vorinostat. If the metastasis has a blood-tumor barrier (BTB), the HDI should be capable of crossing both the BBB and the BTB. Vorinostat is sold under the brand name ZOLINZA® as a treatment for cutaneous T-cell lymphoma as 100 mg capsules. Vorinostat is also known as suberoylanilide hydroxamic acid (SAHA), N-Hydroxy-N′-phenyloctanediamide, and CCRIS 8456. Another suitable HDI is valproic acid (VPA). Valpoic acid, sold under the brand name DEPAKOTE®, has traditionally been administered as an anti-seizure medication to epilepsy patients. However, valproic acid also has activity as a HDI.

The localized carcinoma CNS metastasis of extra-CNS origin can originate from one or more organs in addition to or in the alternative to the breast. Examples of such organs include the lung, colon, liver, and the prostate. Carcinomas are cancers that arise from the epithelium. Aspects of the invention described in respect to carcinoma CNS metastases originating in the breast are also applicable where appropriate to other extra-CNS organs of carcinoma origin. A breast carcinoma CNS metastasis treated in accordance with embodiments of the method of the invention can be derived from a breast ductal carcinoma. In certain embodiments, the breast carcinoma CNS metastasis can be derived from a breast lobular carcinoma.

The subject treated in accordance with the method of the invention can have been diagnosed for breast cancer but need not have been. In some embodiments, the primary breast cancer diagnosed is no longer present. The breast cancer can comprise a genetic signature predictive of metastasis to the brain. The genetic signature can comprise one or more suitable markers. Examples of markers include estrogen receptor (alpha and/or beta) negative phenotype and Her-2 over-expression. Markers can also be based on one or more DNA hypermethylation phenotype such as hypermethylation of cyclin D2, retinoic acid receptor-β and hin-1. Risk factors for brain metastases also include young age and other systemic metastases. The method of treatment of the invention can be begun at any time period following the diagnosis of a primary cancer.

The subject treated in accordance with the method of the invention can have been treated for primary breast cancer and/or breast cancer non-CNS metastases but need not have been. In some embodiments, the subject can have been treated with vorinostat. In certain embodiments, the subject can have been treated with a chemotherapeutic drug other than vorinostat. In some embodiments, the subject can have been further treated with radiation. The treatment can have comprised removal of one or more breast tumors. The subject treated in accordance with the embodiments of the method of the invention can have or have had a carcinoma metastasis in one or more non-CNS organs originating in the breast.

The method of treatment of the invention can be begun at any time period following the diagnosis of a primary cancer or diagnosis of a non-CNS metastasis. In some embodiments, the treatment can be begun at the same time as diagnosis of an earlier, primary cancer. Treatment can begin within 0 hours, 12 hours, 24 hours, 36 hours, 48 hours, 60 hours, 72 hours, 96 hours, one week, two weeks, three weeks, one month, two months, three months, four months, five months, 6 months, 1 year, a year and a half, 2 years, 2 years and a half, 3 years, 4, years, 5 years, 6 years, 10 years 15 years, 20 years, 25 years, 30 years, 40 years, 50 years, 75 years, or more.

The HDI, such as vorinostat, can be administered in accordance with the invention as the sole chemotherapeutic drug. In other embodiments, the vorinostat can be administered in combination with a second chemotherapeutic drug. The administration of the two or more drugs can be simultaneous, sequential or in combination. The second chemotherapeutic drug can be a cytotoxic chemotherapeutic drug. In some embodiments, the second chemotherapeutic drug is not trastuzumab. In some other embodiments, the second chemotherapeutic drug is not tamoxifen. In some other embodiments, the second chemotherapeutic drug is not isotretinoin. In some other embodiments, the second chemotherapeutic drug is not temozolomide. In some embodiments, the second chemotherapeutic drug is temozolomide. The second chemotherapeutic drug can be a different HDI. Other examples of second chemotherapeutic drugs include doxorubicin, methotrexate, flurouracil, carboplatin, and cisplatin. Other non-chemotherapeutic drugs can also be employed. The HDI, such as vorinostat, can be administered in combination with a radiation treatment regimen whether or not additional drugs are employed. The administration of the HDI and radiation can be simultaneous, sequential or in combination. Accordingly, when both a HDI and a second drug or radiation are administered, they need not be administered simultaneously or in the same way or in the same dose. When administered simultaneously, the HDI and the second drug can be administered in the same composition or in different compositions. The HDI and second drug can be administered using the same route of administration or different routes of administration. When administered at different times, the HDI can be administered before or after the second drug or radiation. In some embodiments, administration of the HDI and second drug or radiation is alternated. In certain embodiments, the respective doses of HDI and second drug or radiation are varied over time. The particular HDI can be varied over the treatment period. The particular second drug and/or type of radiation can be varied over the treatment period. When administered at separate times, the separation of the HDI administration and the second drug or radiation administration can be any time period. If administered multiple times, the length of the time period can vary. The separation between administration of HDI and administration of the second drug or radiation can be 0 seconds, 1 second, 5 seconds, 10 seconds, 30 seconds, 1 minute, 5 minutes, 10 minutes, 15 minutes, 20 minutes, 30, minutes, 45 minutes, 1 hour, 1.5 hours, 2 hours, 2.5 hours, 3 hours, 4 hours, 5 hours, 7.5 hours, 10 hours, 12 hours, 15 hours, 18 hours, 21 hours, 24 hours, 1.5 days, 2 days, 3 days, 4 days, 5 days, 6 days, 7 days, 10 days, 2 weeks, 3 weeks, 4 weeks, one month, 6 weeks, 8 weeks, two months, three months, four months, five months, six months, 9 months, 1 year, 2 years, 5, years, 10 years, or an intermediate time period of the preceding. In some embodiments, the therapeutic effect on the carcinoma brain metastatis of administering both the HDI antagonist and drug or radiation is less than additive. In some other embodiments, the therapeutic effect is substantially additive. However, a preferable therapeutic effect is synergistic, that is, more than additive. Accordingly, the HDI and second drug or radiation can be administered in synergistic amounts. The combinatorial effect can be evaluated using any appropriate measurement. Measurements and calculations of synergism can be performed as described in Teicher, “Assays for In Vitro and In Vivo Synergy,” in Methods in Molecular Medicine, vol. 85: Novel Anticancer Drug Protocols, pp. 297-321 (2003).

The subjects treated, screened and otherwise related to the method of the invention can include any suitable living organism. The subject can be a vertebrate animal. The vertebrate can be a fish. The vertebrate can be a bird such as a chicken. The vertebrate can be a mammal. Mammals include, but are not limited to, the order Rodentia, such as mice, the order Logomorpha, such as rabbits, the order Carnivora, including Felines (cats) and Canines (dogs), the order Artiodactyla, including Bovines (cows) and Swines (pigs), the order Perssodactyla, including Equines (horses), and, most preferably, the order Primates, Ceboids, or Simoids (monkeys) or the order Anthropoids (humans and apes). A preferred mammal is the human.

The subject treated in accordance with the method of the invention can have been diagnosed with a carcinoma CNS metastasis and/or susceptible to developing a carcinoma CNS metastasis. In some embodiments, one or more carcinoma CNS metastasis have been detected in the subject. Any appropriate method of detection can be employed. In some embodiments an imaging procedure is employed such as computer aided tomography (CAT) or a magnetic resonance imaging (MRI) scan. Such methods of detection can also be used to follow the effects of the treatment on the subject.

Carcinoma CNS metastases suitable for treatment by the method of the invention can be characterized by morphology, histology, and one or more cell surface macromolecule, e.g., a particular cytokeratin isoform, detection. In some embodiments, the cell surface marker is unique to the carcinoma CNS metastasis relative to the primary systemic cancer from which it originated. In some embodiments, cytokeratin isoforms 18 and/or 19 are characteristic of cancers originating from ductal carcinomas such as breast or colon carcinomas. However, because of the anatomical location of carcinoma CNS metastases, diagnosis generally utilizes a form of imaging such as a CAT or MRI scan.

The treatment of the localized carcinoma brain metastasis can comprise a therapeutic effect on one or more metastasis. Therapeutic effects include, for instance, a reduction of any one or more symptoms or signs (e.g., biological markers) of a carcinoma CNS metastasis. A reduction in a symptom or sign to any degree is considered therapeutic for the purposes of this invention, including, without limitation, the substantial or complete elimination of any such symptoms or signs of the carcinoma CNS metastasis. The specific symptoms and signs that can be reduced or eliminated can depend on the particular carcinoma CNS metastasis being treated. Successful treatment can comprise the elimination of a metastasis, the diminution in volume (shrinking) of a metastasis, reducing the number of metastases, slowing the rate of growth of a metastatsis and/or arresting the growth of a metastasis, a reduction in the rate of spread of a cancer within the CNS after having metastased from outside the CNS, a reduction in the level of expression of one or more cancer markers in a host, and a reduction in the severity or degree of secondary symptoms of the metastasis, such as neurological deficits. Successful outcomes further include stabilizing the metastatic disease and prolonged disease free survival.

In accordance with the method of the invention, the HDI is administered in an amount sufficient to achieve a therapeutically effective concentration in the tissues or fluids of the CNS including the localized carcinoma CNS metastasis. The concentration of HDI that is considered therapeutically effective can depend, in part, upon the particular carcinoma CNS metastasis to be treated, as well as by the severity of the disease and other factors. In some embodiments, a therapeutically effective concentration of the HDI is within the range of about 0.010 nM or more, about 0.10 nM or more, about 10 nM or more, about 15 nM or more, about 20 nM or more, about 30 nM or more, about 40 nM or more, about 60 nM or more, about 80 nM or more, or even about 100 nM or more in the tissues or fluids of the CNS. It certain instances, higher concentrations of one or more HDI may be required, such as about 120 nM or more, about 150 nM or more, about 200 nM or more, about 300 nM or more, about 400 nM or more, about 500 nM or more.

The dose required to achieve a desired concentration of HDI and/or to achieve a given therapeutic effect can be calculated based on the skill in the art in view of the teachings herein, e.g., the in vivo mouse data of Example 5. One of skill in the art can also utilize information available from the FDA website “Drugs@FDA” available at <http://www.accessdata.fda.gov/scripts/cder/drugsatfda/> for commercially available forms of vorinostat, e.g., ZOLINZA®, in such materials as medical, pharmacology, and clinical pharmacology biopharmaceutics reviews for the HDI. The dose of HDI can depend upon the particular carcinoma CNS metastasis being treated as well as the severity of the carcinoma CNS metastasis, the health and fitness of the patient, and various other factors routinely considered by an attending physician. The HDI can be administered in a dose of about 0.010 mg/m² or more, 0.10 mg/m² or more, 1 mg/m² or more, 10 mg/m² or more, 100 mg/m² or more, 200 mg/m² or more, 300 mg/m² or more, 400 mg/m² or more, 500 mg/m² or more, 600 mg/m² or more, 800 mg/m² or more, 1000 mg/m² or more, 1200 mg/m² or more, 1500 mg/m² or more, or 2000 mg/m² or more, which dose can be administered in any suitable regimen (e.g., several times per day (e.g., once, twice, three times, four times, five times, six times, eight times, or ten times per day), daily, every two days, twice per week, once per week, once every two weeks, once per month, etc.). The foregoing dosage amounts can be used as daily dosage amounts, and administered in a single dose (e.g., as an infusion over several minutes (30, 60, 90, or 120 minutes) or several hours (3, 4, 5, or 6 hours), or a single oral dosage) or multiple doses (e.g., multiple infusions in a single day or multiple oral doses). The upper limit of the concentration and dose of HDI used should be less than the level considered to be toxic to the host, and otherwise determined by the concentration needed to treat the particular disease while controlling unwanted side effects.

The HDI, such as vorinostat, and other drugs employed with the methods of the invention can be administered in any suitable form. In some embodiments, the drug or drugs is administered as a prodrug, e.g., an ester, an amide, a salt, a base, an acid, etc. The HDI and other drugs, when CNS-targeted drugs, are administered in sufficient quantity to achieve a therapeutically effective concentration in the CNS.

A therapeutic agent, e.g., a chemotherapeutic drug, which can be a compound and/or a composition, used in accordance with the method of the invention can comprise a small molecule, a nucleic acid, a protein, an antibody, or any other agent with one or more therapeutic property. Examples of chemotherapeutic drugs include HDIs and compositions comprising the same. Examples of HDIs include vorinostat and valproic acid. The therapeutic agent can be formulated in any pharmaceutically acceptable manner. The therapeutic agent that is used in the invention can be formed as a composition, such as a pharmaceutical composition comprising a carrier and a therapeutic compound. Pharmaceutical compositions containing the therapeutic agent can comprise more than one therapeutic compound. The carrier can be any suitable carrier. Preferably, the carrier is a pharmaceutically acceptable carrier. With respect to pharmaceutical compositions, the carrier can be any of those conventionally used and is limited only by chemico-physical considerations, such as solubility and lack of reactivity with the active compound(s), and by the route of administration. In addition to the following described pharmaceutical composition, the therapeutic compounds of the present inventive methods can be formulated as inclusion complexes, such as cyclodextrin inclusion complexes, or liposomes.

The pharmaceutically acceptable carriers described herein, for example, vehicles, adjuvants, excipients, and diluents, are well-known to those skilled in the art and are readily available to the public. The pharmaceutically acceptable carrier can be chemically inert to the active agent(s) and one which has low or no detrimental side effects or toxicity under the conditions of use. The choice of carrier can be determined in part by the particular therapeutic agent, as well as by the particular method used to administer the therapeutic agent. There are a variety of suitable formulations of the pharmaceutical composition of the invention. The following formulations for oral, aerosol, subcutaneous, transdermal, transmucosal, intestinal, parenteral, intramedullary injections, direct intraventricular, intravenous, intranasal, intraocular, intramuscular, intraarterial, intrathecal, intraperitoneal, rectal, and vaginal administration are exemplary and are in no way limiting. More than one route can be used to administer the therapeutic agent, and in some instances, a particular route can provide a more immediate and more effective response than another route. Therapeutic agents can be formulated and administered systemically or locally. Techniques for formulation and administration may be found in Remington's Pharmaceutical Sciences, 18th ed., Mack Publishing Co., Easton, Pa. (1990).

Formulations suitable for oral administration can include (a) liquid solutions, such as an effective amount of the therapeutic agent dissolved in diluents, such as water, saline, or fruit juice such as orange juice; (b) capsules, sachets, tablets, lozenges, dragees, and troches, each containing a predetermined amount of the active ingredient, as solids or granules; (c) powders; (d) suspensions in an appropriate liquid, gel, syrup, or slurry; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard or soft shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise the inhibitor in a flavor, usually sucrose and acacia or tragacanth, as well as pastilles comprising the inhibitor in an inert base, such as gelatin and glycerin, or sucrose and acacia, emulsions, gels, and the like containing, in addition to, such excipients as are known in the art.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

The therapeutic agent, alone or in combination with other suitable components, can be made into aerosol formulations to be administered via inhalation. These aerosol formulations can be placed into pressurized acceptable propellants, such as dichlorodifluoromethane, propane, nitrogen, and the like. They also can be formulated as pharmaceuticals for non pressured preparations, such as in a nebulizer or an atomizer. Such spray formulations also may be used to spray mucosa. Topical formulations can be employed.

Injectable formulations are in accordance with the invention. The parameters for effective pharmaceutical carriers for injectable compositions are well-known to those of ordinary skill in the art [see, e.g., Pharmaceutics and Pharmacy Practice, J.B. Lippincott Company, Philadelphia, Pa., Banker and Chalmers, eds., pages 238 250 (1982), and ASHP Handbook on Injectable Drugs, Toissel, 4th ed., pages 622 630 (1986)]. For injection, the therapeutic agent can be formulated in aqueous solutions, preferably in physiologically compatible buffers such as Hanks's solution, Ringer's solution, or physiological saline buffer. For such transmucosal administration, penetrants appropriate to the barrier to be permeated are used in the formulation. Such penetrants are generally known in the art.

In some embodiments, the therapeutic agent is prepared in a depot form to allow for release to be controlled with respect to time and location within the body (see, for example, U.S. Pat. No. 4,450,150). Depot forms of therapeutic agents can be, for example, an implantable composition comprising the therapeutic agent and a porous or non-porous material, such as a polymer, wherein the therapeutic agent is encapsulated by or diffused throughout the material and/or degradation of the non-porous material. The depot is then implanted into the desired location within the body and the therapeutic agent is released from the implant at a predetermined rate.

Formulations suitable for parenteral administration include aqueous and non aqueous, isotonic sterile injection solutions, which can contain anti oxidants, buffers, bacteriostats, and solutes that render the formulation isotonic with the blood of the intended recipient, and aqueous and non aqueous sterile suspensions that can include suspending agents, solubilizers, thickening agents, stabilizers, and/or preservatives. The therapeutic agent can be administered in a physiologically acceptable diluent in a pharmaceutically acceptable carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, poly(ethyleneglycol) 400, glycerol, dimethylsulfoxide, ketals such as 2,2-dimethyl-1,3-dioxolane-4-methanol, ethers, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations will typically contain from about 0.5% to about 25% by weight of the drug in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

The therapeutic agent can be made into suppositories by mixing with a variety of bases, such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

The exact formulation, route of administration and dosage can be chosen by the individual physician in view of the patient's condition. [See, e.g., Fingl et. al., in The Pharmacological Basis of Therapeutics, 1975, Ch. 1 p. 1]. The attending physician can determine when to terminate, interrupt, or adjust administration due to toxicity, or to organ dysfunctions. Conversely, the attending physician can also adjust treatment to higher levels if the clinical response were not adequate, precluding toxicity. The magnitude of an administrated dose in the management of the carcinoma CNS metastasis can vary with the severity of the disorder to be treated and the route of administration. The severity of the metastasis can, for example, be evaluated, in part, by standard prognostic evaluation methods. The dose and perhaps dose frequency, can vary according to the age, body weight, and response of the individual patient. A program comparable to that discussed above can be used in veterinary medicine.

Therapeutic agents intended to be administered intracellularly can be administered using techniques well known to those of ordinary skill in the art. For example, such therapeutic agents can be encapsulated into liposomes, then administered as described above. Liposomes are spherical lipid bilayers with aqueous interiors. Molecules present in an aqueous solution at the time of liposome formation are incorporated into the aqueous interior. The liposomal contents are both protected from the external microenvironment and, because liposomes fuse with cell membranes, are efficiently delivered into the cell cytoplasm.

The strength of the active ingredient of the therapeutic agent in a particular dosage form can be any appropriate strength. Single or multiple dosages can be taken to achieve the proper dosage. For example, when the dosage form is a tablet, caplet, or capsule, the strength of the active ingredient, e.g., vorinostat, in a particular tablet, caplet, or capsule can be 1 mg or more, 2 mg or more, 5 mg or more, 10 mg or more, 20 mg or more, 50 mg or more, 100 mg or more, 150 mg or more, 200 mg or more, 250 mg or more, 300 mg or more, 350 mg or more, 400 mg or more, 450 mg or more, 500 mg or more, 600 mg or more, 700 mg or more, 750 mg or more, and 1 g or more. In some embodiments, the therapeutic agent employed is the vorinostat formulation ZOLINZA® brand 100 mg capsules. In some embodiments, the therapeutic agent is a vorinostat formulation analogous to ZOLINZA® brand 100 mg capsules but with a greater or lesser amount of vorinostat.

The invention also provides for the use of a HDI in the manufacture of a medicament for the treatment of localized carcinoma CNS metastasis of extra-CNS origin. Accordingly, the invention provides a HDI for use in treatment of a localized carcinoma CNS metastasis of extra-CNS origin. Additionally, the invention provides a medicinal formulation comprising a HDI for treating localized carcinoma CNS metastasis of extra-CNS origin.

The following examples further illustrate the invention but, of course, should not be construed as in any way limiting its scope.

### EXAMPLE 1

This example demonstrates that there are differences in gene expression between malignant epithelial cells from brain metastases and those from a non-related cohort of primary invasive breast tumors. These differences can be exploited to design cancer therapies based on the down regulation of genes in cancer cells.

Laser capture microdissection (LCM) is used to isolate the malignant epithelial cells from sixteen brain metastatic lesions from breast cancer patients and sixteen non-related primary breast tumors, which are listed in Table 1. A total of at least 10 ng of total RNA is isolated and amplified to generate between 50-100 μg of amplified antisense RNA from cDNA microarray analysis. In all experiments, a six cell line pool of breast cancer cells is used as a common reference sample. Cy3- or Cy5-dUTP labeled cDNA (Amersham Pharmacia Biotech) is synthesized from 50 μg of RNA using random primed polymerization with Superscript II reverse transcriptase (Life Technologies). Equal amounts of Cy incorporated cDNA for the test sample and the reference sample are hybridized to a 30k cDNA array for each tumor analyzed. Fluorescent intensities are measured using a GenPix scanner and scanned images are analyzed using DeArray software.

TABLE 1
Cohort Characteristics

| | Brain Metastases | Primary Tumors |
|---|---|---|
| n | 16 | 16 |
| Patient age | 36-68 | 45-73 |
| Primary tumor classification | | |
| Ductal | 9 | 11 |
| Lobular | 1 | 1 |
| Inflammatory | 2 | 0 |
| ER status of primary tumor | | |
| Positive | 5 | 7 |
| Negative | 7 | 5 |
| TNM stage | | |
| T1N0M0 | 1 | 3 |
| T1N1M0 | | 1 |
| T1N2M0 | 1 | 0 |
| T1Nx | 1 | 1 |
| T2N0M0 | 4 | 2 |
| T2N1M0 | 4 | 2 |
| T2N0Mx | 0 | 1 |
| T2Nx | 0 | 1 |
| T3N1M0 | 1 | 0 |
| T3N2Mx | 0 | 1 |

Eight brain metastases and nine primary tumors are analyzed by microarray and significance analysis of microarray (SAM) software (<http://www-stat.stanford.edu/˜tibs/SAM/>) is used to generate a pseudocolor heatmap of genes that significantly distinguish the brain metastasis from the primary tumors. Genes up-regulated and down-regulated in the brain metastases relative to the primary tumors are listed in Tables 2A and 2B. Quantitative real-time PCR (Q-PCR) is used to confirm the gene expression differences noted in the microarray analysis. Four metastases and four primary tumors from the original cohort (two ER-positive and two ER-negative for both groups) along with the remaining eight brain metastasis and eight primary tumors are analyzed for a subset of genes that differentiate the cohorts to validate the microarray results. Samples are independently prepared by LCM and amplified before being reverse transcribed for PCR analysis. A majority of genes are downregulated in the brain metastases cohort compared to the non-related cohort of primary invasive breast tumors as shown in Tables 2A and 2B.

TABLE 2A
Up-Regulated Genes

| | Symbol | M | Name |
|---|---|---|---|
| 1 | HK2 | 1.90446533 | Hexokinase 2 |
| 2 | PTPLB | 1.70088784 | PTPLB: protein tyrosine phosphatase-like member b |
| 3 | LAMC3 | 0.79328291 | Laminin gamma-3 |
| 4 | HSRTSBETA | 1.21685051 | |
| 5 | YY1 | 0.64938583 | |
| 6 | PIGL | 0.73897576 | phosphatidylinositol glycan, class L |
| 7 | DCLRE1C | 1.50575258 | DNA cross-link repair 1C (PSO2 homolog, S. |
| 8 | ATPIF1 | 0.77968815 | ATPase inhibitory factor 1 |
| 9 | ASB1 | 0.84770298 | ankyrin repeat and SOCS box-containing 1 |
| 10 | SHB | 0.97196112 | (Src homology 2 domain containing) adaptor protein B |
| 11 | WAC | 0.8083518 | WW domain containing adaptor with coiled-coil |
| 12 | CASK | 0.97595849 | calcium/calmodulin-dependent serine protein kinase |
| 13 | PPFIA1 | 1.12983907 | protein tyrosine phosphatase, receptor type, f |
| | | | interacting protein (liprin), alpha 1 |
| 14 | ZIC1 | 4.02149155 | Zic family member 1 |
| 15 | PIGA | 0.81852402 | phosphatidylinositol glycan, class A |

TABLE 2B
Down-Regulated Genes

| | | | |
|---|---|---|---|
| 16 | ADAM12 | −2.8510692 | ADAM12: a disintegrin and metalloproteinase |
| | | | domain 12 |
| 17 | FST | −1.595219 | Follistan |
| 18 | PLEKHA4 | −0.848892 | pleckstrin homology domain containing, family |
| | | | A member 4 |
| 19 | ADAM12 | −2.7018589 | |
| 20 | STMN3 | −1.5431747 | Stathmin-like 3 |
| 21 | RARRES2 | −1.6882617 | retinoic acid receptor responder 2 |
| 22 | LOXL1 | −1.1382505 | Lysyl-oxidase-like 1 |
| 23 | COL8A2 | −1.5115524 | Collagen 8 |
| 24 | COL15A1 | −1.9151285 | Collagen 15 |
| 25 | SLIT3 | −1.3299513 | |
| 26 | CAPG | −1.3514448 | Capping protein, glesolin-like |
| 27 | LOXL1 | −0.9991855 | |
| 28 | POMT1 | −0.7345072 | protein-O-mannosyltransferase 1 |
| 29 | FST | −1.3786612 | |
| 30 | RAB31 | −1.2978479 | |
| 31 | THSD2 | −1.8314645 | thrombospondin, type I, domain containing 2 |
| 32 | SRPX | −1.514539 | sushi-repeat-containing protein, X-linked |
| 33 | CILP | −2.7841428 | cartilage intermediate layer protein, nucleotide |
| | | | pyrophosphohydrolase |
| 34 | COL15A1 | −2.0844783 | |
| 35 | GAS1 | −2.9479555 | Growth Arrest-specific 1 |
| 36 | SPOCK | −1.467814 | testican- osteonectin, cwcv and kazal-like |
| | | | domains proteoglycan |
| 37 | TM4SF7 | −0.8562885 | transmembrane 4 superfamily member 7 |
| 38 | BHC80 | −0.6356215 | BRAF35/HDAC2 complex (80 kDa |
| 39 | MLL4 | −0.7337228 | myeloid/lymphoid or mixed-lineage leukemia 4 |
| 40 | SPIB | −0.7995737 | Spi-B transcription factor (Spi-1/PU.1 related) |
| 41 | BAL | −1.0124637 | PARP9: poly (ADP-ribose) polymerase family, |
| | | | member 9 |
| 42 | MRC2 | −0.9439598 | mannose receptor, C type 2 |
| 43 | MFAP4 | −0.6911835 | microfibrillar-associated protein 4 |
| 44 | SERPINF1 | −2.0169322 | pigment epithelium derived factor |
| 45 | CYP3A4 | −1.3798621 | cytochrome P450, family 3, subfamily A, |
| | | | polypeptide 4 |
| 46 | TRIM34 | −0.6224236 | tripartite motif-containing 34 |
| 47 | PCDH16 | −0.7158834 | dachsous 1 |
| 48 | NMB | −0.9292313 | neuromedin B |
| 49 | POSTN | −3.5141777 | periostin, osteoblast specific factor |
| 50 | BMP1 | −1.3675727 | bone morphogenetic protein 1 |
| 51 | MMP2 | −2.6927657 | matrix metalloproteinase 2 |
| 52 | SIAH2 | −1.1952985 | seven in absentia homolog 2 |
| 53 | CCL2 | −1.737577 | chemokine (C-C motif) ligand 2 |
| 54 | MEOX1 | −0.6072613 | mesenchyme homeo box 1 |
| 55 | COL1A2 | −2.0979478 | Collagen 1 |
| 56 | SF1 | −0.4947576 | splicing factor 1 |
| 57 | TNFAIP2 | −1.3785244 | tumor necrosis factor, alpha-induced protein 2 |
| 58 | RGS16 | −0.7478252 | regulator of G-protein signalling 16 |
| 59 | UNC5B | −0.9104926 | unc-5 homolog B |
| 60 | DNM1 | −1.0309777 | Dynamin |

### EXAMPLE 2

This example demonstrates that HDI treatment of breast cancer cells predisposed for metastasis to the brain alter the expression of metastasis associated genes and increases acetylation of histones in the same. Treatment of a brain metastatic subline of the MDA-MB-231 (231-BR) breast carcinoma cell line with HDAC inhibitors alters the gene expression of numerous genes disregulated in the brain metastases cohort described in Example 1.

The human MDA-MB-231BR “brain seeking” (231-BR) cell line used is described in Yoneda et al., J. Bone and Mineral Res. 16, 1486-1495 (2001). All cell lines are deemed free of mycoplasma and human pathogens and test negative in mouse antibody production (MAP) tests. The cells are treated with 5 μM SAHA for 0, 8, 24, and 48 hours prior to lysis. Assays are done for other compounds at the same set of durations including depsipeptide (10 ng/mL), valproic acid (VPA) (10 mM), trichostatin (TSA) (100 ng/mL). Drug solution is added once at the beginning and left on the cells for the indicated duration (0, 8, 24, and 48 hours). Controls are performed in the same way with the same amount of vehicle solution for each drug, but without the drug. The vehicle can be DMSO. Cells are lysed according to standard procedures and Western blotting is performed. Primary antibodies specific to the targeted proteins are used including antibodies specific to acetyl Histone H3, acetyl histone H4, p21, and α-tubulin. Horseradish peroxidase-conjugated secondary antibodies purchased from Santa Cruz Biotechnology (Santa Cruz, Calif.) are used at dilutions of 1:5000. Proteins are visualized using enhanced chemilluminescence (Cell Signaling) and autoradiography. Western blots of lysates of cells treated for 0, 8, 24, 48 with depsipeptide at 10 ng/mL or SAHA at 5 μM. Targeted proteins include acetyl Histone H3, acetyl histone H4, p21, and tubulin. As histones H3 and H4 become hyperacetylated, protein levels of the cyclin-dependent kinase inhibitor p21 increase. Those changes indicate HDAC inhibition.

A heatmap of the 231-BR cell gene expression changes upon treatment with HDIs, including depsipeptide and SAHA, is prepared. The map includes visualization of RNA extracted at timepoints of 0, 24, and 48 hours of HDI treatment. Microarray analysis is performed and the top 500 genes altered by HDAC inhibition (comparison of 24 or 48 hour treatment with time 0) are represented. The heatmap microarray results are validated by Western blot analysis of proteins whose expression is induced or repressed by SAHA, depsipeptide, or valproic acid treatment. The target proteins in the Western blot include gelsolin, TSP-1, CTGF, CDK5, cyclin B1, cyclin B2 and tubulin. Microarrayanalysis of 231BR cells “top” down-regulated proteins restored by SAHA treatment are shown in Table 3.

TABLE 3

| | Fold- |
|---|---|
| Gene | Induction |
| PLEKHA4 (Pleckstrin homology domain containing) | 1.68 |
| STMN3 (Stathmin like 3) | 1.60 |
| LOXL1 (Lysyl oxidase like) | 1.58 |
| MRC2 (Mannose receptor C type 2) | 2.93 |
| FHOD3 (Formain homology 2 domain) | 2.15 |
| SERPINF1 (PEDF) | 2.30 |
| POMT1 (Protein-O-mannosyl transferase 1) | 1.58 |
| BF (Filensin) | 2.36 |
| SPIB (Spi transcription factor) | 1.88 |
| SHB | 1.90 |
| SPRX (Sushi-repeat containing protein) | 1.52 |
| BAL (B aggressive lymphoma) | 1.54 |
| PCDH16 (Proto-cadherin 16) | 1.82 |

### EXAMPLE 3

This example demonstrates that HDIs inhibit proliferation of breast cancer cells predisposed for metastasis to the brain. 231-BR Cells are used as described in Example 2. The prepared cells are plated at a density of 15,000 cells/well in a 96 well plate and incubated for 3 hours to permit attachment. Cells are then washed with PBS, and media containing either 0.1% or 1% FBS is added to the cells. Drug concentrations used are as described in Example 2. After a 72-hour incubation, 0.5 mg/mL MTT (Sigma, St. Louis, Mo.) is added and plates are incubated for 2 hours. Media is then aspirated, and MTT dissolved in DMSO for 30 minutes, after which the absorbance is read at a wavelength of 570 nm. The absorbance recorded on day 3 was divided by the absorbance recorded on day 0 (day of plating), and results are displayed as fold growth compared to day 0 control in Table 4. Data in Table 4 is shown as percent of colonies formed±standard deviation compared to untreated controls. Results are representative of three independent experiments in quintuplicate. Analysis of variance (ANOVA) is used to assess in vitro functions of vehicle treated versus vorinostat treated cells. P values can be two-tailed. Tests can be performed using GraphPad InStat version 3.0 software.

TABLE 4
Colonization Data

| | % Control |
|---|---|
| Control | 100 ± 8 |
| SAHA | 47 ± 1.9 |
| Depsipeptide | 10 ± 2.5 |
| Trichostatin (TSA) | 30 ± 3.4 |
| Valporic Acid | 46 ± 5.3 |

Both depsipeptide and SAHA treatment of 231BR cells results in growth inhibition when sufficient HDI is administered. SAHA is applied to 231BR cells in various amounts from 0 to 100 μM, absorbance is measured at 570 nm, and absorbance plotted against SAHA (μM) yielding an IC₅₀ of 12.6 μM and a dose of 5 μM. Noticeable inhibition occurs at 1 μM and higher doses of SAHA. Depsipeptide is applied to 231BR cells in various amounts from 0 to 10 μg/mL, absorbance is measured at 570 nm, and absorbance plotted against depsipeptide yielding an IC₅₀ of 1.5 μg/mL and a dose of 0.01 μg/mL. Noticeable inhibition occurs at 0.001 μg/mL and higher doses of depsipeptide.

### EXAMPLE 4

This example demonstrates that HDIs inhibit chemotaxis of breast cancer cells predisposed for metastasis to the brain. 231-BR Cells are used as described in Example 2. A 48-well Boyden chemotaxis chamber is used. Polycarbonate PVP-free Nucleopore filters (8 μm pore size) are coated with 0.01% collagen (BD Bioscience). FBS (1%) in DMEM with 1 mg/ml BSA is used as the chemoattractant in the lower chamber. Drug concentrations used are as described in Example 2. 231-BR cells, after 24 hours of vorinostat, other drug treatment, or control are added to the top chamber in DMEM with 1 mg/ml BSA at a concentration of 2×10⁶ cells/ml. Chambers are incubated for 4 hours in a 37° C. incubator with 5% CO₂. After chambers are disassembled, filters are fixed and stained with reagents from a Diff-Quik® Kit (Fischer Scientific). Cells that migrate through the Boyden chamber are counted using a light microscope. Representative areas are counted to determine the number of cells that have migrated for each well. Results are shown in Table 5. Data in Table 5 are shown as mean number of cells±standard deviation that migrate per well. Analysis of variance (ANOVA) is used to assess in vitro functions of vehicle treated versus vorinostat treated cells. P values can be two-tailed. Tests can be performed using GraphPad InStat version 3.0 software.

TABLE 5
Motility Data

| | Control | 0.5% FCS |
|---|---|---|
| Control | 613 ± 118 | 2540 ± 185 |
| SAHA | 67 ± 6 | 756 ± 56 |
| Depsipeptide | 129 ± 23 | 678 ± 33 |
| TSA | 108 ± 13 | 785 ± 17 |
| Valporic Acid | 242 ± 55 | 755 ± 37 |

### EXAMPLE 5

This example demonstrates that a BBB-crossing HDI, such as vorinostat, can successfully treat localized carcinoma CNS metastases in mammals when administered systemically.

Cells are prepared as described in Example 2 with additional preparation as follows. The retroviral vector pLEGFP-C1 (BD Biosciences, San Jose, Calif.) is transfected into murine fibroblast PT67 packaging cells using Effectene reagent according to the manufacturer's protocol (Qiagen, Germantown, Md.). After 24 hours, enhanced green fluorescent protein (EGFP)-expressing cells are selected in the presence of 1 mg/mL G418 (Invitrogen, Carlsbad, Calif.), and colonies are expanded. Virus is harvested and filtered through a 0.45 um Millex-HA syringe-driven filter (Millipore, Billerica, Mass.) and 231-BR cells are infected with retrovirus for 6 hours. The following day, 231-BR cells are selected in the presence of 0.8 mg/mL G418, and EGFP expression in 95-99% of the cells is confirmed by fluorescent microscopy.

All animal experiments are conducted under an approved Animal Use Agreement with the NCI. Under isoflurane anesthesia, 20 female Balb/c nude mice (Charles River Laboratories, Frederick, Md.) 5-7 weeks old are inoculated with 500,000 (Experiment 1) or 100,000 (Experiment 2) MDA-MB-231-BR cells in 0.1 mL PBS in the left ventricle of the heart. Mice are monitored daily for signs of ill health. Three days after tumor cell inoculation mice are randomized to treatment groups and treatment is started. SAHA is administered via intraperitoneal (IP) injection once daily 7 days a week for 21 days. The drug is injected in a solution of 10% DMSO and 45% PEG400 in water and the same solution minus SAHA is used for the vehicle control group. After 21 days of treatment, mice are euthanized under CO₂ anesthesia and brains are excised for imaging. EGFP is detected in whole brains by the Maestro 420 In Vivo Spectral Imaging System (Cambridge Research and Instrumentation, Woburn, Mass.), using software (e.g., Nuance Technology, Burlington, Mass.) to distinguish or unmix images of fluorescence from multiple sources. After imaging, mouse brains are bisected along the sagittal plane and the right hemisphere of the brain is fixed in 4% paraformaldehyde for 24-48 hours at 4° C., then transferred to 20% sucrose overnight at 4° C. and frozen (for EGFP detection/histology). The left hemisphere is forallin-fixed and paraffin embedded for immunohistochemistry. Ten micron brain sections are serially cut and processed for either EGFP microscopy or histology. To detect EGFP, slides are dried at room temperature and 25 uL Vectashield HardSet Mounting Media with DAPI was added (Vector Laboratories, Burlingame, Calif.). Images are captured on a Zeiss Axioskop 2 microscope (Thornwood, N.Y.) using OpenLab software (Improvision, Lexington, Mass.). For staining with hematoxylin and eosin, a standard protocol is followed. Ten serial sections every 300 microns through the brain are analyzed using a 5× objective on a Zeiss microscope, containing an ocular grid with squares of 0.8 mm².

Every micro- or large (>50 microns²) metastasis in each section is tabulated. Data are representative of two experiments conducted. Data for the two experiments are shown in Tables 6 and 7 respectively. Wilcoxon rank sum test is used to compare the number of total metastases and the number of large metastases in vehicle treated versus vorinostat treated mice. Tests can be performed using GraphPad InStat version 3.0 software. “Mean Micromets” in Tables 6 and 7 refer to the mean number of metastases counted in 10 step sections from one hemisphere of the brain. “Mean Large Mets” in Tables 6 and 7 refer to the size of metastases determined by a 16 mm² ocular grid. Large metastases are greater than 50 microns². In Experiment 1, on about the eighth day, i.e., after about 5 days of SAHA treatment, the higher dosage is lowered from 200 mg/kg to 150 mg/kg due to toxicity.

TABLE 6
Vorinostat Experiment 1

| mg/kg | Number | Mean | | Mean | |
|---|---|---|---|---|---|
| SAHA | of mice | Micromets | 95% CI | Large Mets | 95% CI |
| 0 (Vehicle) | 5 | 205.5 | 175-236 | 6.8 | 5.6-7.7 |
| 150 mg/kg | 6 | 151.8 | 112-191 | 3.5 | 2.5-4.5 |
| 100 mg/kg | 3 | 140.4 | 112-169 | 2.9 | 0.2-5.6 |

TABLE 7
Vorinostat Experiment 2

| mg/kg | Number | Mean | | Mean | |
|---|---|---|---|---|---|
| SAHA | of mice | Micromets | 95% CI | Large Mets | 95% CI |
| 0 (Vehicle) | 5 | 72.5 | 51-100 | 3.3 | 2.6-3.9 |
| 150 mg/kg | 9 | 66.6 | 56-77 | 1.5 | 1.1-1.8 |
| 100 mg/kg | 9 | 78.3 | 53-109 | 4.4 | 3.5-5.3 |

Accordingly, in vivo, the HDAC inhibitor vorinostat reduces the number of large (>50 micron²) brain metastases.

### EXAMPLE 6

This example further demonstrates that vorinostat (SAHA) can successfully treat carcinoma CNS metastases in mammals when administered systemically.

Given the significant reduction in metastatic outgrowth observed with administration of 150 mg/kg SAHA (see Example 5), the effect of the timing of SAHA administration is tested. As described in Example 5, 175,000 MDA-MB-231BR cells in 0.1 mL PBS are injected into the left cardiac ventricle of the heart of nude mice. Starting on days 3, 7, or 14 post-injection, vehicle or 150 mg/kg SAHA is administered via IP injection once daily until day 21 post-injection. SAHA is injected in a solution of 10% DMSO and 45% PEG400 in water and the same solution minus SAHA is used for the vehicle control group. On day [X], mice are euthanized under CO₂ anesthesia and brains are excised for imaging as described in Example 5.

Micro- and large (>50 microns²) metastases are tabulated. The data set forth in Table 8 are representative of two experiments.

TABLE 8

| | | Micrometastases | | | Large Metastases | | |
|---|---|---|---|---|---|---|---|
| | | Mean | | | Mean | | |
| | | number | | | number | | |
| | Number | per | | | per | | |
| Treatment | of mice | section | 95% CI | P value | section | 95% CI | P value |
| Vehicle | 20 | 170 | 146-193 | | 7.65 | 6.20-9.10 | |
| 150 mg/kg | 18 | 122 | 98-146 | 0.017 | 2.89 | 1.94-3.84 | <0.0001 |
| SAHA starting | | | | | | | |
| on day 3 post- | | | | | | | |
| injection | | | | | | | |
| 150 mg/kg | 19 | 151 | 127-176 | NS | 4.94 | 3.90-5.98 | 0.008 |
| SAHA starting | | | | | | | |
| on day 7 post- | | | | | | | |
| injection | | | | | | | |
| 150 mg/kg | 18 | 171 | 153-201 | NS | 5.96 | 4.69-7.22 | NS |
| SAHA starting | | | | | | | |
| on day 14 post- | | | | | | | |
| injection | | | | | | | |

NS = not significantly different

Administration of SAHA starting on day 3 post-injection results in a 57% reduction in large metastases (P<0.001), which confirms the efficacy data set forth in Example 5. A 28% reduction in micrometastases also is observed, which achieves statistical significance (P=0.017).

By delaying SAHA administration until day 7 post-injection, the efficacy of SAHA is reduced, although treatment with SAHA remains statistically significantly different from vehicle treatment (34% reduction, P=0.008).

If SAHA is administered on day 14 post-injection, only a 22% reduction in large metastases is observed, which is not statistically significant.

The data suggest that early use of SAHA will be most advantageous in the treatment of carcinoma CNS metastases of extra-CNS origin.

### EXAMPLE 7

This example describes the further characterization of the effect of SAHA on localized carcinoma CNS metastases of extra-CNS origin.

Proliferation of the brain metastases in vehicle-treated and 150 mg/ml SAHA-treated mice is assessed by Ki67 staining. For the detection of Ki-67, immunostaining is performed with the anti-Ki-67 mouse monoclonal antibody (clone MIB-1, DakoCytomation, CA), which labels Ki67 antigen in the granular components of the nucleolus during late G1, S, G2 and M phases. Detection of Ki67 antigen in neoplastic cell populations is used to assess cell proliferation.

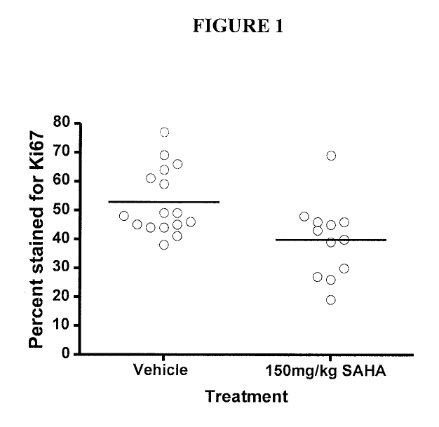

Both micrometastases and large metastases are highly proliferative with approximately 50% of lesions staining. Treatment with SAHA results in a minor reduction in the Ki67 staining of large metastases (see FIG. 1), which indicates a reduction in cell proliferation in SAHA-treated metastases as compared to vehicle-treated metastases. No effect is observed on micrometastases.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating a localized carcinoma central nervous system (CNS) metastasis of extra-CNS origin, the method comprising systemically administering an effective amount of a histone deacetylase (HDAC) inhibitor (HDI) to a subject in need of treatment for the localized carcinoma CNS metastasis of extra-CNS origin.

2. The method of claim 1, wherein the HDI is vorinostat.

3. The method of claim 1, wherein the localized carcinoma CNS metastasis of extra-CNS origin is a localized carcinoma brain metastasis.

4. The method of claim 3, wherein the localized carcinoma brain metastasis is located in one or more tissues selected from the group consisting of brain parenchyma and the leptomeninges.

5. The method of claim 3, wherein the localized carcinoma brain metastasis of extra-CNS origin originated in one or more organs selected from the group consisting of the lung, breast, colon, liver and prostate.

6. The method of claim 3, wherein the localized carcinoma brain metastasis of extra-CNS origin originated in the breast and the localized carcinoma brain metastasis of extra-CNS origin is a localized breast carcinoma brain metastasis.

7. The method of claim 6, wherein the breast carcinoma brain metastasis is derived from a breast ductal carcinoma.

8. The method of claim 6, wherein the breast carcinoma brain metastasis is derived from a breast lobular carcinoma.

9. The method of claim 1, wherein the subject has been diagnosed for primary breast cancer.

10. The method of claim 9, wherein the primary breast cancer comprises a genetic signature predictive of metastasis to the brain.

11. The method of claim 10, wherein the genetic signature comprises one or more markers selected from the group consisting of estrogen receptor negative and Her-2 over-expression.

12. The method of claim 9, wherein the subject has been treated for primary breast cancer.

13. The method of claim 12, wherein the subject has been treated with a chemotherapeutic drug other than vorinostat.

14. The method of claim 12, wherein the subject has been further treated with radiation.

15. The method of claim 12, wherein the subject has been further treated by removal of the primary breast tumor.

16. The method of claim 1, wherein the subject has or has had a further carcinoma metastasis in one or more non-CNS organs originating in the breast.

17. The method of claim 2, wherein the vorinostat is administered as the sole chemotherapeutic drug.

18. The method of claim 2, wherein the vorinostat is administered in combination with a second chemotherapeutic drug.

19. The method of claim 18, wherein the second chemotherapeutic drug is a cytotoxic chemotherapeutic drug.

20. The method of claim 18, wherein the second chemotherapeutic drug is not trastuzumab.

21. The method of claim 18, wherein the second chemotherapeutic drug is not tamoxifen.

22. The method of claim 18, wherein the second chemotherapeutic drug is not isotretinoin.

23. The method of claim 18, wherein the second chemotherapeutic drug is not temozolomide.

24. The method of claim 2, wherein the vorinostat is administered in combination with a radiation treatment regimen.

25. The method of claim 1, wherein the subject is human.

26. The method of claim 1, wherein the brain metastasis comprises a micrometastasis, a brain tumor, or an intervening stage of brain cancer.

27-29. (canceled)
